(19) United States
(12) Patent Application Publication
(10) Pub. No.: US 2003/0071970 A1
(43) Pub. Date: Apr. 17, 2003

(54) Ophthalmologic examination instrument

(75) Inventors: Karl-Heinz Donnerhacke, Jena (DE); Egon Luther, Cospeda (DE); Ingo Koschmieder, Jena (DE)

Correspondence Address: Gerald H. Kiel, Esq., REED SMITH LLP, 599 Lexington Avenue, New York, NY, 10022-7650, US

(73) Assignee: Carl Zeiss Meditec AG

(21) Appl. No.: 10/268,319

(22) Filed: Oct. 10, 2002

(30) Foreign Application Priority Data
Oct. 17, 2001 (DE) 101 51 314.3

(51) Int. Cl. A61B 3/10

(52) U.S. Cl. 351/221

(57) ABSTRACT

Microperimetry and examinations of the front and back of the eye are made possible by the ophthalmologic examination instrument. The ophthalmologic examination instrument with at least one illumination arrangement for generating temporally and spatially variable light marks and/or luminous fields has an input unit for adjusting the illumination conditions, a signaling device for reporting the detectability and/or undetectability of the light marks, an output unit, and a control unit. In the method, a light mark for fixating the patient's eye and a movable light mark which is variable with respect to position, shape, brightness and/or color is projected in sequence on the back of the eye. Conclusions can be made about the vision of the patient from the detectability of the light marks on the part of the patient. Based on its varied possibilities for producing and manipulating light marks and/or luminous fields, the suggested solution offers an extremely broad applicability in eye examinations. It is possible to locate spatially small functional disturbances on the back of the eye in a fast and reliable manner through monitored microperimetry while simultaneously observing the fundus and with the participation of the subject.

### CROSS-REFERENCE TO RELATED APPLICATIONS

This application claims priority of German Application No. 101 51 314.3, filed Oct. 17, 2001, the complete disclosure of which is hereby incorporated by reference.

### BACKGROUND OF THE INVENTION

a) Field of the Invention

The present invention is directed to an ophthalmologic examination instrument by means of which a determination of the visual field and microperimetric examinations of patients can be carried out in addition to the examination of the front portion and back portion of the eye.

b) Description of the Related Art

Various ophthalmologic examination instruments are known from the prior art, each of which was conceived for specific examinations at or in the eye.

For perimetry, for example, there are ophthalmologic instruments which determine the visual field of a subject by simulation using light marks. For this purpose, an entire raster of test points is distributed over the visual field to be examined and a sensitivity measurement is carried out at all of these points. The smaller the raster, the smaller the defects that can be found in the visual field. Partial fields of particular interest can be examined with an extremely fine raster. Computer perimeters of different manufacturers are described more fully in [1] below with reference to their technical data. A disadvantage in these instruments is that they are generally not suitable for other diagnostic examinations. The apparatus-related costs for a complex eye examination involving examination of the fundus with determination of the visual field and discovery of visual field defects are correspondingly high as a result of the different devices needed for this purpose.

Current perimeters have the disadvantage that because the retina is examined point by point and not over the whole surface it is often difficult to discover spatially small or unclear functional disturbances and to assign the latter to local causes in the eye.

The ophthalmologic microscopes known from the art, e.g., slit lamps, are used in normal operation for examination of the front portion of the eye. The examined area can be expanded to the back portion of the eye according to [1] by means of additional contact lenses or lenses (e.g., Volk lenses or Hruby lenses). However, they are unsuitable or only conditionally suitable for perimetry. A special illumination unit is used in slit lamps to generate a changeable slit imaging. A light section is generated by slit image projection in the eye being examined. The parameters of this section bundle are variable, particularly with respect to the angle of incidence, the dimensioning of the slit image, its intensity and its spectral composition. Conclusions may be reached about the state of the individual media of the eye from the shape, position and intensity of the scattered light of the sectional image generated in this way. Inspection of the fundus with the slit lamp is a frequently used method common in practice. As is shown in [2] below, a perimetric examination is also possible in principle when a correspondingly small punctiform light mark can be generated.

In slit lamps such as those described in [1], mechanical/optical elements such pinhole and slit diaphragms, filter glasses, test patterns, and so on, were formerly used to vary the luminous field geometry. These mechanical component groups are very cumbersome to adjust, with the added difficulty caused by the thermal expansion of the component groups. Reproducibility of adjustments for measurement purposes is only possible to a limited extent. The variety of possible luminous field geometries is extremely limited by the fixed slit diaphragms and the space requirement.

Arrangements of the type mentioned above have the further disadvantage that the shape and size of the light marks that can be generated by modem slit lamps are not optimized with respect to the requirements of perimetry. Light marks are still predominantly generated by mechanical diaphragms whose variability and quantity in the instrument are limited. Another substantial disadvantage consists in that the position of the light marks on the back of the eye can not be changed conveniently or to a sufficient extent for tracking or illuminating the contours of specific retinal areas without changing the basic adjustment of the instrument.

DE 198 12 050 A1 describes a method and an arrangement for illumination in an ophthalmologic microscope in which a large variety of luminous mark geometries are generated by optoelectronic components. The luminous field geometries are projected on the front of the eye or on the back of the eye and used for general examination of the eye.

### REFERENCES

[1] Rassow, B., et al., “Ophthalmologisch-optische Instrumente [Ophthalmologic Optical Instruments]”, 1987, Ferdinand Enke Verlag, Stuttgart, pages 99 ff. and 137 ff.

[2] Mojon, D. S., “Die Spaltlampen-Perimetrie [Slit lamp perimetry]”, “Der Augenspiegel”, 7-8/2000, pages 20 ff.

### OBJECT AND SUMMARY OF THE INVENTION

It is the primary object of the present invention to develop an ophthalmologic instrument so as to make possible a general examination of the fundus (front of the eye and back of the eye) and determination of the visual field of patients without extensively altering the instrument construction.

According to the invention, this object is met by an ophthalmologic examination instrument with an observation system, various beam-shaping and deflection elements and at least one illumination arrangement for generating optically, temporally and spatially variable light marks and/or luminous fields on the back of the eye in that there is an input unit for selecting and setting the illumination conditions to be adjusted, a signaling device allowing the patient to signal the detectability and/or undetectability of the light marks, an output unit and a control unit for controlling the optoelectronic components and overall process and for storing data. In the method for determining the visual field of patients, a light mark for fixating the patient's eye and a light mark which is variable in position, shape, brightness and color are projected on the back of the eye in sequence. Conclusions can be made about the visual field of the patient from the detectability of the image information with respect to the position, shape and brightness of the light marks.

Based on its varied possibilities for producing and manipulating light marks and/or luminous fields, the suggested technical solution for an ophthalmologic instrument offers an extremely broad applicability in eye examinations. It can be used for examinations of the fundus as well as for determining the visual field and for the most common examinations carried out on the human eye. In particular, the suggested solution is suitable for microperimetry, i.e., for a spatially limited perimetry, specifically while simultaneously examining the fundus.

The invention will be described more fully in the following with reference to an embodiment example.

### BRIEF DESCRIPTION OF THE DRAWINGS

In the drawings:

FIG. 1 shows a possible basic construction of the arrangement according to the invention with a DMD microdisplay;

FIG. 2 shows another possible basic construction of the arrangement, according to the invention, with an LCOS microdisplay; and

FIG. 3 shows a variable light mark projected on the retina with coordinate system and background illumination.

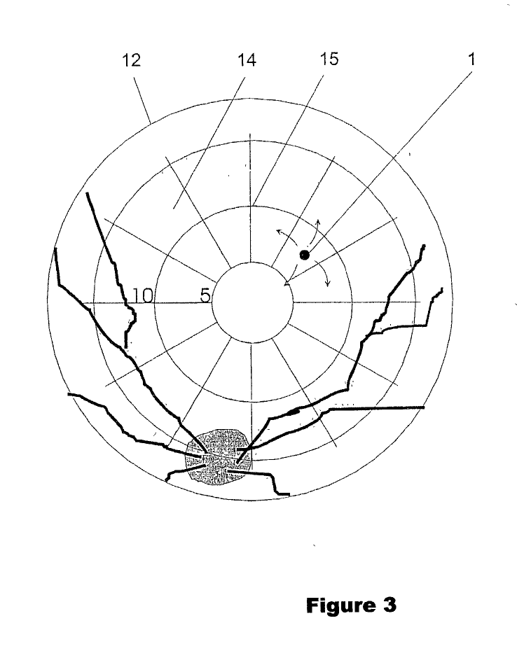

### DESCRIPTION OF THE PREFERRED EMBODIMENTS

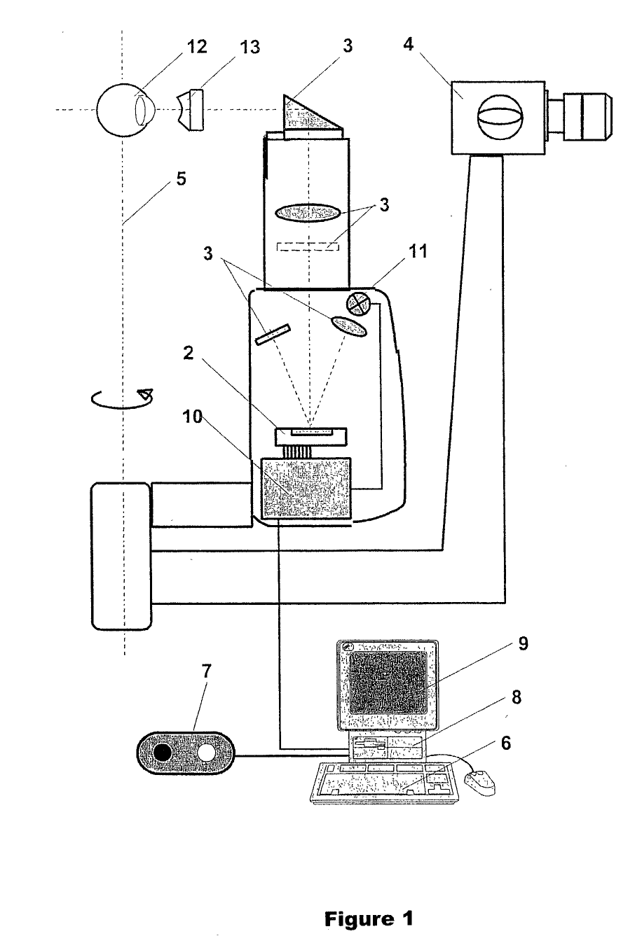

The ophthalmologic examination instrument shown in FIG. 1 is essentially a slit lamp in which the optoelectronic component 2 provided as illumination arrangement for generating variable light marks 1 and/or luminous fields is provided with individually controllable pixel elements of a microdisplay, e.g., a DMD microdisplay, as an individual or additional illumination unit. A DMD (digital mirror device) microdisplay has individually controllable micromirrors. In a known manner, at least one observation system 4 and various beam-shaping and deflecting elements 3 are provided. The ophthalmologic examination instrument comprises an observation system 4 and an illumination system. The two systems are swivelable about the axis of rotation 5 independent from one another. Further, the ophthalmologic examination instrument has an input unit 6 for selecting and setting the illumination conditions to be adjusted, a signaling device 7 which signals the detectability and/or undetectability of the light marks on the part of the patient, and a control unit 8 for controlling the optoelectronic components 2 and overall process and for storing the data. The control unit 8, for which a PC can be used, for example, has connections to the input unit 6, the signaling device 7, an output unit 9 and, via interfaces 10, to the optoelectronic component 2 and light source 11. The connections can be wire connections or can also be produced in a wireless manner. A keypad, control lever, ball, touchpad, PC mouse, speech-controlled unit, remote control, micromanipulator or other suitable arrangements can be used as input unit 6 for selecting and setting the illumination conditions to be adjusted.

In the method for the examination and determination of the visual field of patients and particularly for microperimetric examination by means of the described ophthalmologic examination instrument, a light mark for fixating the patient's eye 12 and the variable light mark 1 required for perimetry are projected on the back of the eye. This is carried out in that the optoelectronic component 2 is illuminated by an illumination source 11. The conditions for the optically, temporally and spatially variable light mark 1 which is generally punctiform with any, preferably small, diameter are preset by the control unit 8. By means of a commercially available contact lens 13 or additional lens, e.g., a Volk lens, the light mark 1 is projected onto the retina of the eye 12 being examined. By displacing it on the retina, this light mark 1 can be used to find areas with functional disturbances, e.g., scotomas. The manipulation of the light mark 1 is carried out via the input unit 6 or operator control at the ophthalmologic instrument. The direction of displacement can advantageously be determined from the responses of the patient about the individual visibility of the light mark 1. In contrast, the visual field is determined, as a rule, in a fully program-controlled manner and the data of the variable light marks 1 projected in random sequence are stored for evaluation in connection with the detectability or undetectability reported by the patient by means of a signaling unit 7. The signaling unit 7 can be a hand button or foot button, a speech-controlled unit, a unit for evaluation of brain currents or an arrangement corresponding to the input unit 6. As a rule, the stored data are outputted as a result of visual field determination in the form of sensitivity profiles. The output unit 9 for tracking the course of the examination and for displaying the results of the examination can be a monitor, a printer or an HMD (head mounted display) according to DE 197 20 851. For a repeat examination of the patient, it can be advantageous when the sequence of variable light marks 1 projected on the back of the eye is stored together with coordinates or other information that can serve for finding a specific examination area again more quickly for an examination which may possibly be repeated.

It is advantageous for perimetry and particularly for microperimetry as well as for examination of the fundus of a patient's eye 12 to project a background illumination 14 and/or a coordinate system 15 on the back of the eye in addition to the light marks. This is advantageously carried out by means of the optoelectronic components 2. The background illumination 14 and/or the coordinate system 15 are likewise variable with respect to shape, brightness, color and spatial and temporal position. The selection of the parameters in their entirety, including the spectral composition, should be made in such a way that the patient is not influenced or dazzled.

The spectral composition of the light marks 1 and/or luminous fields can be determined and varied by controlling the optoelectronic components 1, by filters additionally arranged between the illumination source 11 and optoelectronic component 2 or by the illumination source 11 itself.

For eye examinations of longer duration, it is also advantageous when the light mark projected on the back of the eye for fixation and the background illumination and/or coordinate system track the patient's eye movement. This can be carried out based on prominent points on the retina (e.g., the network of blood vessels), The patient's capacity for concentration can accordingly be substantially increased compared to examinations with permanently stationary fixating marks.

In addition to an observation system 4, modem ophthalmologic examination instruments also generally have an image processing unit by means of which images of the eye can be recorded and processed. The image of the eye is projected onto a CCD matrix, for example, by means of additional beam splitters.

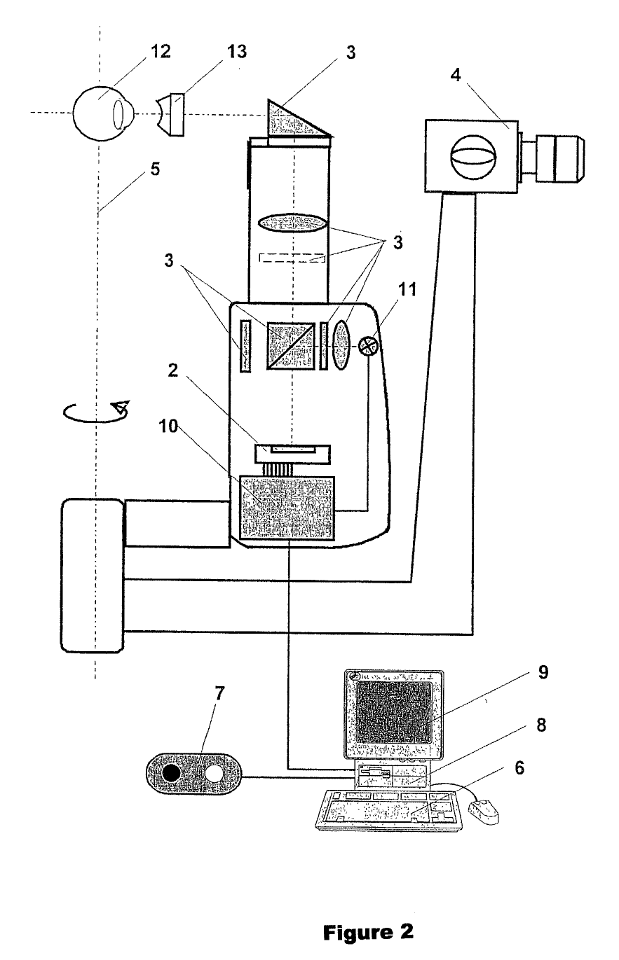

The ophthalmologic examination instrument according to FIG. 2 is also essentially a slit lamp in which the optoelectronic component 2 serving as illumination arrangement for generating the variable light marks 1 and/or luminous fields is provided with individually controllable pixel elements of a microdisplay, e.g., a LCOS microdisplay, as an individual or additional illumination unit. A LCOS (liquid crystal on silicon) microdisplay has LCD cells which are individually controllable with respect to transmissivity with polarized light. With the exception of the optoelectronic component 2 and the associated polarization optics, the essential construction corresponds to that described in FIG. 1. Since the individual process steps are also identical, reference is had to the description of the method according to FIG. 1.

Another construction variant, not shown, provides for the use of an LCD or LED type optoelectronic component 2. The LCD (liquid crystal display) microdisplay also has LCD cells which are individually controllable with respect to transmissivity in polarized light. However, the construction is changed in such a way that the LCD type optoelectronic component 2 is to be operated in transmitted light mode and associated polarization optics are required. The LED (light emitting diode) microdisplay and particularly the OLED (organic light emitting diode) microdisplay likewise comprise individually controllable pixel elements which, in contrast to the optoelectronic components 2 described above, emit light themselves. This makes it possible to simplify the construction by omitting the light source and polarization optics. However, the individual process steps are identical to the arrangements already described.

The method according to the invention and the arrangement suitable for carrying out this method make it possible to locate spatially small functional disturbances on the back of the eye in a fast and reliable manner through monitored microperimetry while simultaneously observing the fundus. Perimetry and fundoscopy can be combined with respect to time in such a way that only one device is required for this purpose. Accordingly, as distinct from a separate examination, an exact and direct correlation of lesions and scotomas is possible, for example.

While the foregoing description and drawings represent the present invention, it will be obvious to those skilled in the art that various changes may be made therein without departing from the true spirit and scope of the present invention.

What is claimed is:

1. An ophthalmologic examination instrument comprising:
an observation unit;
at least one illumination arrangement for generating temporally and spatially variable light marks and/or luminous fields on the back of the eye, illumination being carried out proceeding from a light source by optoelectronic components for specific beam deflection;
an input unit for selecting and setting the illumination conditions to be adjusted;
a control unit for controlling the optoelectronic components and overall process and for storing data; and
an output unit;
said control unit having connections for the input unit to said output unit for tracking the examination process and/or for displaying the results of the examination.

2. The ophthalmologic examination instrument according to claim 1, wherein the input unit for selecting and setting the illumination conditions to be adjusted is a keypad, a control lever, a manipulator, a ball, a touchpad, a PC mouse, a remote control, or a speech-controlled unit.

3. The ophthalmologic examination instrument according to claim 1, wherein microdisplays with individually controllable pixel elements or microscanner mirrors are used as optoelectronic components.

4. The ophthalmologic examination instrument according to claim 1, wherein the optoelectronic components are controlled manually, automatically or by program.

5. The ophthalmologic examination instrument according to claim 1, wherein a signaling device is provided for reporting the detectability and/or undetectability of the light marks on the part of the patient.

6. The ophthalmologic examination instrument according to claim 1, wherein the signaling device is a hand button, foot button, remote control, keypad, control lever, manipulator, ball, touchpad, PC mouse or a unit for evaluating brain currents.

7. The ophthalmologic examination instrument according to claim 1, wherein the output unit is a monitor or printer, or in that an HMD (head mounted display) is used as output unit.

8. The ophthalmologic examination instrument according to claim 1, wherein the control unit is a PC which is connected via interfaces to the other components or in that the control unit is a computer which is integrated in the ophthalmologic examination instrument.

9. A method for the examination and determination of the visual field of patients, particularly for operation of an ophthalmologic examination instrument according to claim 1, including the steps of:
projecting a light mark for fixating the patient's eye onto the back of the eye;
projecting a light mark which is variable with respect to shape, brightness, color and spatial and/or temporal position in sequence on the back of the eye;
controlling the sequence of this variable light mark by input elements or a stored process, sending an associated signal to the control unit by the patient in response to the detectability or undetectability of the respective light mark;
making conclusions about the vision of the patient from the detectability of the image information with respect to position, shape and brightness of the light marks;
storing the data of the variable light marks for evaluation in connection with the detectability or undetectability reported by the patient; and
outputting the stored, processed or prepared data as a result of the examination, particularly the determination of the visual field.

10. The method for the examination and determination of the visual field of a patient according to claim 9, wherein the variable light mark is punctiform.

11. The method for the examination and determination of the visual field of a patient according to claim 9, wherein a background illumination and/or a coordinate system are/is projected onto the back of the eye in addition to the light marks, in that the background illumination and/or a coordinate system projected onto the back of the eye in addition to the light marks are/is variable with respect to shape, brightness, color and spatial and temporal position, and in that the light marks for fixation and the background illumination and/or the coordinate system projected on the back of the eye follow the eye movement of the patient.

12. The method for the examination and determination of the visual field of a patient according to claim 9, wherein the sequence of variable light marks is random with respect to position, shape, brightness and color, or in that the sequence of the variable light marks is controlled depending on the signals supplied by the patient, wherein the random or arbitrary sequence of variable light marks can be carried out accompanied by observation; and
wherein the sequence of variable light marks projected on the back of the eye are stored together with coordinates or other information that can serve for finding a specific examination area again more quickly for a process which may possibly be repeated.

13. The method for the examination and determination of the visual field of a patient according to claim 9, wherein the evaluation is carried out by means of an automatic image evaluation.

14. The method for the examination and determination of the visual field of a patient according to claim 9, wherein a manual or automatic examination sequence, e.g., for process control, can be repeated based on the stored data.
